(19) United States
(12) Patent Application Publication
(10) Pub. No.: US 2006/0253040 A1
(43) Pub. Date: Nov. 9, 2006

(54) Method and device for measuring systolic and diastolic blood pressure and heart rate

(75) Inventors: Stergios Stergiopoulos, Toronto (CA); Dimitrios Hatzinakos, Scarborough (CA)

Correspondence Address: FREEDMAN & ASSOCIATES, 117 CENTREPOINTE DRIVE, SUITE 350, NEPEAN, ONTARIO, K2G 5X3, CA

(73) Assignee: Canamet Canadian National Medical Technologies Inc, Toronto (CA)

(21) Appl. No.: 11/356,958

(22) Filed: Feb. 21, 2006

Related U.S. Application Data
(60) Provisional application No. 60/656,382, filed on Feb. 28, 2005.

(51) Int. Cl. A61B 5/02

(52) U.S. Cl. 600/493; 600/490

(57) ABSTRACT

A method and a device for measuring blood pressure and heart rate is disclosed. Blood pressure signals corresponding to the Korotkoff sounds are detected using an array of primary acoustic sensors. A secondary acoustic sensor is used for detecting noise and vibrations. The signals provided by the primary acoustic sensors are then processed using adaptive linear beamforming and are combined into a single signal. The combined signal is then provided to an adaptive interferer canceller together with the signal of the secondary acoustic sensor for removing noise and vibration effects. Artifacts due to a patient's motion are removed in a following post-processing step. The post-processed signal and the pressure signal are then graphically displayed enabling a trained user to correct the automatic measurement, to decide whether a repeat measurement is necessary, and to quickly and accurately diagnose heart pulse patterns.

This application claims benefit from U.S. Provisional Patent Application No. 60/656,382 filed Feb. 28, 2005 the entire contents of which are incorporated herein by reference.

### FIELD OF THE INVENTION

This invention relates generally to the field of blood pressure measurement methods and devices and more particularly to auscultatory blood pressure measurement methods and devices employing means for removing noise and vibration effects as well as artifacts due to a patient's motion from audible blood flow sounds.

### BACKGROUND OF THE INVENTION

The blood pressure in the brachial artery is not constant, but varies with time in relation to the beating of the heart. Following a contraction of the heart to pump blood through the circulatory system, the blood pressure increases to a maximum level known as the systolic blood pressure. The minimum blood pressure between heartbeats is known as the diastolic blood pressure.

The traditional auscultatory technique for measuring the blood pressure of a patient employs an inflatable pressure cuff wrapped around an upper arm of a patient whose blood pressure is to be determined. As the pressure cuff is inflated, cuff pressure and pressure applied to the arm of the patient increases. If the pressure applied to the arm is increased beyond the highest blood pressure in the brachial artery located in the arm beneath the pressure cuff, the artery is forced to close.

As the pressure in the inflatable cuff is reduced from a high level above the systolic blood pressure, where the brachial artery is permanently closed, to a level below the systolic blood pressure level, the brachial artery beneath the cuff will begin to open and close with each heart beat as the blood pressure first exceeds the cuff pressure and then falls below the cuff pressure. As the blood pressure exceeds the cuff pressure, the artery will open, and a low frequency blood pressure sound, the so-called “Korotkoff sound” is detectable. This sound is detected using a bell of a stethoscope or microphone placed over the brachial artery near the down-stream end of the cuff on the patient's arm. The highest cuff pressure at which the Korotkoff sounds are detectable thus corresponds to the systolic blood pressure of the patient.

As the cuff pressure is reduced further, the cuff pressure will be brought below the diastolic blood pressure. At this pressure level, the brachial artery beneath the cuff remains open throughout the heart beat cycle. Blood pressure sounds, caused by the opening of the artery will, therefore, not be produced. The lowest cuff pressure at which the blood pressure sounds can be detected thus corresponds to the diastolic blood pressure of the patient.

In manual auscultatory blood pressure measurement methods, a stethoscope is used to detect the onset and disappearance of the Korotkoff sounds. Thus, the blood pressure measurement is highly dependent on the skill and hearing ability of the person taking the measurement. To overcome this dependence on human skill and judgement, and to automate the process of determining a patient's blood pressure, automatic blood pressure measurement systems based on the auscultatory method of blood pressure determination have been developed.

However, it is almost impossible to detect the Korotkoff sounds in a noisy environment such as a moving ambulance, helicopter, airplane, or naval vessel. A substantial improvement in accuracy of automated blood pressure measurements based on the auscultatory method in noisy environments has been achieved by employing adaptive beamforming and adaptive interference cancellation as disclosed by Stergios Stergiopoulos et al. in U.S. Pat. No. 6,805,671 issued Oct. 19, 2004.

Furthermore, the Korotkoff sound signal comprises substantial artifacts that arise during travel of the Korotkoff sound signal through a patient's body or from physiological motion of the patient. These artifacts also appear in the Korotkoff sound signal when the blood pressure is measured in a “quiet” environment such as a doctor's office or clinic. Unfortunately, the adaptive interference cancellation is not able to reduce such artifacts.

Another aspect to be considered in automated blood pressure measurement systems is the possibility of errors: human error such as incorrect placement of the pressure cuff, or errors of the automated system such as conditions of the Korotkoff signal which result in incorrect processing of the signal. However, in many situations it is difficult for a user to detect an incorrect measurement based only on a numerical result, in particular in stress situations encountered, for example, by a paramedic in an ambulance.

It would be advantageous to provide for measuring blood pressure based on the auscultatory method with reduced artifacts.

Furthermore, it would be advantageous to provide a graphical presentation of the measurements emulating the manual auscultatory method for enabling a medical practitioner to graphically verify the measurement.

### SUMMARY OF THE INVENTION

It is, therefore, an object of the invention to provide a method and a device for measuring systolic and diastolic blood pressure in noisy environments comprising a signal processing step for reducing artifacts caused by a patient's motion.

It is further an object of the invention to provide a graphical representation of the measurements emulating the manual auscultatory method.

In accordance with the present invention there is provided, a method for determining systolic and diastolic blood pressure of a patient comprising:
receiving Korotkoff data indicative of a Korotkoff sound signal sensed during deflation of a pressure cuff wrapped around a limb of the patient;
receiving pressure signal data indicative of cuff pressure during the deflation of the pressure cuff;
processing the received Korotkoff data comprising:

determining a basic period corresponding to a heart pulse period;

dividing the Korotkoff data into non-overlapping segments of a length corresponding to approximately the basic period;

determining for each segment a metric;

calculating a statistical indicator of the determined metrics;

comparing for each segment the metric of the segment to the statistical indicator producing a comparison result for each segment;

producing post-processed Korotkoff data by comparing for each segment the comparison result to a predetermined threshold and if the comparison result is above the predetermined threshold replacing the signal data of the segment with the statistical indicator; and,

determining the systolic and the diastolic blood pressure based upon the post-processed Korotkoff data and the received pressure signal data.

In accordance with an aspect of the present invention there is provided, a method for determining systolic and diastolic blood pressure of a patient comprising:
providing to a processor Korotkoff data indicative of a Korotkoff sound signal sensed during deflation of a pressure cuff wrapped around a limb of the patient;
providing to the processor pressure signal data indicative of cuff pressure during the deflation of the pressure cuff;
using the processor determining the systolic and the diastolic blood pressure based upon the Korotkoff data and the received pressure signal data; and,

using a display in communication with the processor plotting data based on the Korotkoff data and the pressure signal data in a same graph and indicating data points in the graph corresponding to the determined systolic and diastolic blood pressure.

In accordance with the present invention there is further provided, a storage medium having stored therein executable commands for execution on a processor, the processor when executing the commands performing:
receiving Korotkoff data indicative of a Korotkoff sound signal sensed during deflation of a pressure cuff wrapped around a limb of the patient;
receiving pressure signal data indicative of cuff pressure during the deflation of the pressure cuff;
processing the received Korotkoff data comprising:

determining a basic period corresponding to a heart pulse period;

dividing the Korotkoff data into non-overlapping segments of a length corresponding to the basic period;

determining for each segment a metric;

calculating a statistical indicator of the determined metrics;

comparing for each segment the metric of the segment to the statistical indicator producing a comparison result for each segment;

producing post-processed Korotkoff data by comparing for each segment the comparison result to a predetermined threshold and if the comparison result is above the predetermined threshold replacing the signal data of the segment with the statistical indicator; and,

determining the systolic and the diastolic blood pressure based upon the post-processed Korotkoff data and the received pressure signal data.

In accordance with the present invention there is yet further provided, a device for determining systolic and diastolic blood pressure of a patient comprising:
at least a primary acoustic sensor for being placed on skin of a limb of the patient over an artery occluded by applying pressure thereupon using a pressure cuff, each of the at least a primary acoustic sensor for producing a primary acoustic sensor signal in dependence upon Korotkoff sounds during deflation of the pressure cuff;
a pressure transducer for sensing the cuff pressure during the deflation of the pressure cuff and for providing a pressure signal in dependence thereupon;
a processor in communication with the at least a primary acoustic sensor and the pressure transducer, the processor for performing:

determining a basic period corresponding to a heart pulse period;

dividing the Korotkoff data into non-overlapping segments of a length corresponding to the basic period;

determining for each segment a metric;

calculating a statistical indicator of the determined metrics;

comparing for each segment the metric of the segment to the statistical indicator producing a comparison result for each segment;

producing post-processed Korotkoff data by comparing for each segment the comparison result to a predetermined threshold and if the comparison result is above the predetermined threshold replacing the signal data of the segment with the statistical indicator; and,
determining the systolic and the diastolic blood pressure based upon the post-processed Korotkoff data and the received pressure signal.

In accordance with the aspect of the present invention there is further provided, a device for determining systolic and diastolic blood pressure of a patient comprising:
a primary acoustic sensor for being placed on skin of a limb of the patient over an artery occluded by applying pressure thereupon using a pressure cuff, the primary acoustic sensor for producing a primary acoustic sensor signal in dependence upon Korotkoff sounds during deflation of the pressure cuff;
a pressure transducer for sensing the cuff pressure during the deflation of the pressure cuff and for providing a pressure signal in dependence thereupon;
a processor in communication with the primary acoustic sensor and the pressure transducer for receiving primary acoustic sensor data in dependence upon the primary acoustic sensor signal and pressure signal data in dependence upon the pressure signal, the processor for performing:
determining the systolic and the diastolic blood pressure based upon the primary acoustic sensor data and the pressure signal data;
determining graphical data for plotting the primary acoustic sensor data and the pressure signal data in a same graph and indicating points in the graph related to the determination of the systolic and diastolic blood pressure; and,
a display in communication with the processor for receiving the graphical data and displaying the same.

### BRIEF DESCRIPTION OF THE DRAWINGS

Exemplary embodiments of the invention will now be described in conjunction with the following drawings, in which:

FIG. 1 is a simplified block diagram of a device for measuring systolic and diastolic blood pressure according to the invention;

FIG. 2a is a simplified flow diagram of a signal processing structure according to the invention;

FIG. 2b is a simplified flow diagram of a basic processing step according to the invention of the signal processing structure illustrated in FIG. 2a;

FIGS. 3a to 3e illustrate results of the signal processing steps shown in FIG. 2a;

FIG. 4 is a schematic diagram illustrating the determination of the diastolic and systolic blood pressure;

FIG. 5 illustrates a graphical user interface according to the invention for displaying the blood pressure measurement results;

FIGS. 6a and 6b illustrate a situation where the blood pressure measurement missed the systolic blood pressure and the corrected result using the graphical user interface according to the invention;

FIGS. 7a and 7b illustrate two cases of special heart beat patterns—Whistle and Arrhythmia—using the graphical user interface according to the invention; and,

FIGS. 8a and 8b are simplified block diagrams illustrate two embodiments of a device for measuring systolic and diastolic blood pressure according to the invention.

### DETAILED DESCRIPTION

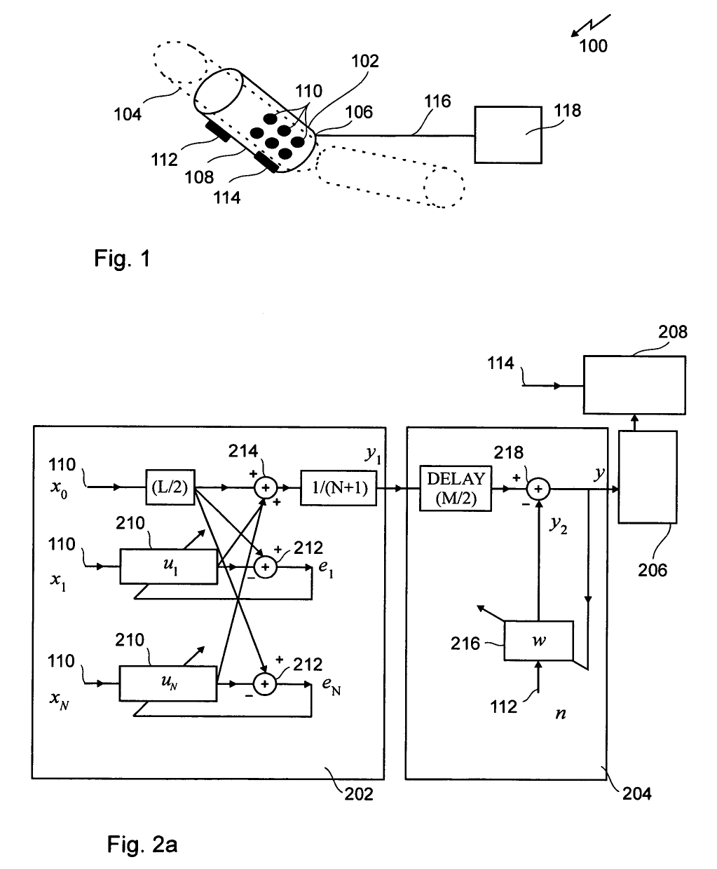

FIG. 1 illustrates schematically a preferred embodiment of a device 100 for measuring systolic and diastolic blood pressure based on the auscultatory method according to the invention. The device 100 comprises an inflatable pressure cuff 102 to be wrapped around a limb 104 of a patient whose blood pressure is to be determined. When wrapped around the patient's limb 104 the pressure cuff 102 substantially forms a cylinder having an inside surface 106 and an outside surface 108. Mounted to the inside surface 106 is at least a primary acoustic sensor 110, preferably an array of primary acoustic sensors 110. The array of primary acoustic sensors 110 is located on the inside surface 106 of the pressure cuff 102 for positioning on the skin of the patients limb near the brachial artery at the downstream end of the pressure cuff 102 with respect to blood flow in the brachial artery. The primary acoustic sensors 110 comprise, for example, microphones placed in a bell shaped housing, and are disposed for capturing Korotkoff sounds and for providing primary acoustic sensor signals in dependence thereupon. A secondary acoustic sensor 112 is mounted to the outside surface 108 of the pressure cuff 102 facing away from the brachial artery in order to capture noise and vibrations of the surrounding environment which are superposed to the Korotkoff sounds and, therefore, detected by the primary acoustic sensors 110, and for providing a secondary acoustic sensor signal in dependence thereupon. A pressure transducer 114 is disposed inside the pressure cuff 102 for measuring cuff pressure, i.e. pressure exerted by the inflated pressure cuff 102 onto the brachial artery, and for providing a pressure signal in dependence thereupon. The primary acoustic sensors 110, the secondary acoustic sensor 112 and the pressure transducer 114 are connected via a communication link 116 to a housing 118 comprising means such as an A/D converter and a processor for processing the primary acoustic sensor signals, the secondary acoustic sensor signal and the pressure signal.

In operation the pressure cuff 102—wrapped around an upper arm of the patient—is inflated to a pressure beyond the highest blood pressure in the brachial artery forcing the brachial artery to close. The pressure cuff 102 is inflated manually or by a motor driven pump.

As the pressure in the inflatable cuff 102 is reduced to a level below the systolic blood pressure level, the brachial artery beneath the cuff will begin to open and close with each heart beat as the blood pressure first exceeds the cuff pressure and then falls below the cuff pressure. The arterial wall acts in a non-linear fashion with respect to the blood pressure level. Thus, as the blood pressure exceeds the cuff pressure, the artery will open, producing low frequency blood pressure sounds—Korotkoff sounds—corresponding to the heart beat. This sound is then detected using the primary acoustic sensors 110. Therefore, the pressure detected by the pressure transducer 114 at the time instance when a first Korotkoff sound is detected corresponds to the systolic blood pressure.

As the cuff pressure is reduced further, the cuff pressure will fall below the diastolic blood pressure. At this pressure level, the brachial artery beneath the cuff remains open throughout the heart beat cycle. Korotkoff sounds, caused by the opening of the artery will, therefore, not be produced. The lowest cuff pressure at which the Korotkoff sounds are detected thus corresponds to the diastolic blood pressure.

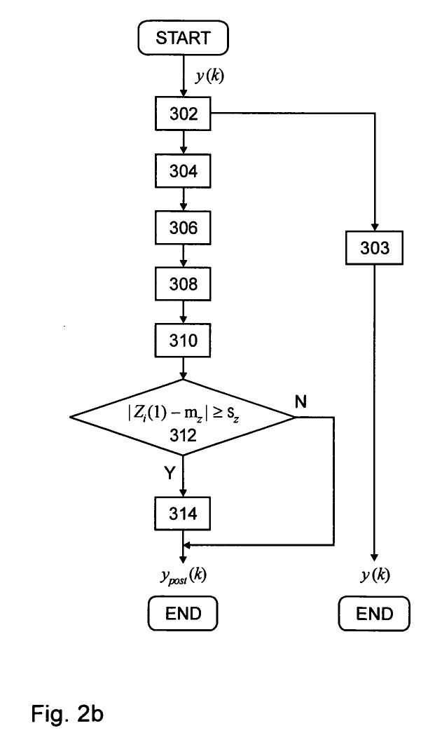

Referring to the block diagram shown in FIG. 2a, a preferred embodiment of a method according to the invention for processing the signals produced by the device 100 is shown. The signal processing method comprises the four basic steps of:

adaptive linear beamforming 202,

adaptive noise/interference cancellation 204,

post-processing for additional artifact reduction 206, and

blood pressure/pulse rate determination 208.

Digitized primary acoustic sensor signals x₀(k),x₁(k), . . . x_{N}(k)—with k=1, 2, . . . , K indicating sample number of the digitized time dependent signals—from the primary acoustic sensors 110 (0) to 110(N) are received in block 202 for adaptive linear beamforming and combining. The sensor signal x₀(k) is delayed by L/2 for use as reference in the following filtering process, with L+1 being a length of each of recursive filters 210. The length of the recursive filters 210 depends on the positions of the primary acoustic sensors 110 relative to each other and the sampling rate used in the digitization of the sensor signals, but is generally substantially less than a heart beat period.

Each of the digitized primary acoustic sensor signals x₁(k),x₂(k), . . . x_{N}(k) is processed using one of the recursive linear filters 210 with a filter coefficient vector uᵢ^{k}=[uᵢ^{k}(−L/2), . . . ,uᵢ^{k}(0), . . . ,uᵢ^{k}(L/2)], i=1, . . . , N, respectively. The filter coefficient vector uᵢ^{k}=[uᵢ^{k}(−L/2), . . . ,uᵢ^{k}(0), . . . , uᵢ^{k}(L/2)] is adjusted using a gradient optimization process, which is well known in the art. At iteration step k+1 the filter coefficient vector is determined as:
uᵢ^{k+1}=uᵢ^{k}+μ₁(k)·eᵢ(k)·xᵢ^{k} (1)
where xᵢ^{k}=[xᵢ(k−L/2), . . . ,xᵢ(k), . . . ,xᵢ(k+L/2)], the step size μ₁(k)=1((xᵢ^{k})(xᵢ^{k})^{H}), with H denoting conjugate transpose operation, and

$${e}_{i} ( k ) \cdot = {x}_{0} ( k - L / 2 ) - {\sum }_{j = - L / 2}^{L / 2} {x}_{i} ( k + j ) \cdot {u}_{i}^{k} ( j ) \qquad ( 2 )$$

with equation (2) being performed in node 212. The output of the block 202 for the k^{th} sample is

$${y}_{1} ( k ) = \frac{1}{N + 1} ( {x}_{0} ( k - L / 2 ) - {\sum }_{i = 1}^{N} {\underline{x}}_{i}^{k} \cdot {\underline{u}}_{i}^{{k}^{T}} ) \qquad ( 3 )$$

where T denotes transpose operation. Referring to the block diagram shown in FIG. 2a, the terms xᵢ^{k}·uᵢ^{k}^{^{T}} are determined in each of the respective recursive linear filters 210 and then summed and subtracted from the delayed reference sensor signal x₀(k−L/2) in node 214. In other words, the above process in block 202 minimizes a difference between the reference acoustic sensor signal and each of the other N primary acoustic sensor signals and adds them coherently in order to achieve a total array gain of 10 log (N) at its output.

In the following processing step the output signal y₁(k) of block 202 is then provided to block 204 to remove interference due to noise and vibrations in the surrounding environment sensed by the secondary acoustic sensor 112 using an adaptive interference canceller (AIC). The AIC is an effective method for removing interference due to noise and vibrations surrounding a patient from a primary acoustic sensor signal, provided there is no correlation between the primary acoustic sensor signal and the secondary acoustic sensor signal, as disclosed by Stergios Stergiopoulos et al. in U.S. Pat. No. 6,805,671 issued Oct. 19, 2004.

The output signal y₁(k) received from processing block 202 is delayed by M/2 and provided to node 218, with M+1 being a length of adaptive filter 216. The digitized secondary acoustic sensor signal n(k) is processed using the adaptive filter 216 with a canceller coefficient vector w^{k}=[w^{k}(−M/2), . . . ,w^{k}(0), . . . ,w^{k}(M/2)]. The canceller coefficient vector w^{k}=[w^{k}(−M/2), . . . ,w^{k}(0), . . . ,w^{k}(M/2)] is adjusted in the adaptive filter 216 using a gradient optimization process, which is well known in the art. At iteration step k+1 the canceller coefficient vector is determined as:
w^{k+1}=w^{k}+μ₂·y(k)·n^{k} (4)
where n^{k}=[n(k−M/2), . . . ,n(k), . . . , n(k+M/2)], and the step size μ₂(k)=1/((n^{k})(n^{k})^{H}). The output of the block 204 for the k^{th} sample is

$$\begin{matrix}y ( k ) \cdot = {y}_{1} ( k - M / 2 ) - {y}_{2} ( k ) \\ = {y}_{1} ( k - M / 2 ) - {\sum }_{j = - M / 2}^{M / 2} n ( k + j ) \cdot {w}^{k} ( j )\end{matrix} \qquad ( 5 )$$

where y₂(k) is determined in the adaptive filter 216 and then subtracted from the delayed signal y₁(k−M/2) in the node 218.

Alternatively, the AIC process is applied to each primary acoustic sensor signal x₁(k),x₂(k), . . . x_{N}(k) prior the step of combining of the signals, as outlined above with respect to block 202. However, performing the AIC after combining the primary sensor signals is the more efficient implementation and does not sacrifice accuracy since the above combining process is a linear operation.

Unfortunately, the AIC process is not able to reduce artifacts arising during travel of the primary acoustic signal through a patient's body or due to physiological motion of the patient. Therefore, the output signal y(k) from block 204 is post-processed in block 206 for artifact reduction as illustrated in the corresponding flow diagram shown in FIG. 2b. The artifact post-processing according to the invention utilizes the repeatability and periodicity properties of the sensed signal—the Korotkoff sounds.

First, an estimate of a basic period corresponding to a heart pulse period P of the signal y(k), k=0, 1, . . . , K−1 is obtained—box 302—by calculating an autocorrelation function R_{y}(m) and finding a second major peak with:

$${R}_{y} ( m ) \cdot = \frac{1}{K} {\sum }_{k = max ( 0 , - m )}^{min ( K , K - m )} y ( k ) \cdot y ( k + m ) , \\ m = 0 , 1 , … , M \qquad ( 6 )$$

where

$$M \approx \frac{K}{3} .$$

Since the signal y(k) has an inherent periodicity corresponding to the heart pulse period P, the autocorrelation function R_{y}(m) exhibits peaks at multiples of the heart pulse period P. Since the autocorrelation function always has its maximum at m=0, it is the second peak of the autocorrelation function R_{y}(m) that corresponds to the heart pulse period P. It is noted, that in case of small to moderate Arrhythmia equation (6) still provides an average value of the heart pulse period P. If, however, the Arrhythmia is severe the process will fail and the post-processing is abandoned—box 303. Optionally, the user is notified about the abandonment and the digitized signal y(k) is provided to a graphical display enabling a trained user to determine a patient's condition.

Second, the mean m_{y} is removed from the signal y(k)—box 304—and the signal is then divided into non-overlapping segments of length P—box 306—as follows:

$$\begin{matrix}{m}_{y} = \frac{1}{K} {\sum }_{k = 0}^{K - 1} y ( k ) & ( 7 ) \\ \tilde{y} ( k ) = y ( k ) - {m}_{y} , k = 0 , 1 , … , K - 1 & ( 8 ) \\ {z}_{i} ( j ) = \tilde{y} [ ( i - 1 ) \cdot P + j ] , j = 0 , 1 , … , P - 1 , i = 1 , … , [ K / P ] & ( 9 )\end{matrix}$$

where [K/P] indicates the integer part of K/P.
Third, for each segment a metric such as a magnitude Zᵢ(1) of the second coefficient of the P point FFT of a segment envelope is calculated—box 308, i.e.

$${Z}_{i} ( 1 ) = \vert {\sum }_{n = 0}^{P - 1} \vert {z}_{i} ( n ) \vert \cdot {ⅇ}^{- j 2 π n / P} \vert , i = 1 , … , [ K / P ] \qquad ( 10 )$$

The motivation for calculating such a metric stems from the periodic nature of the Korotkoff sounds, which is expected to be approximately the same over intervals of one period. The above metric corresponds to the average power of a first harmonic of the signal.

Fourth, a statistical indicator such as an average m_{z} and the variance σ_{z}² of the obtained metrics Zᵢ(1) are calculated—box 310—as:

$$\begin{matrix}{m}_{z} = \frac{1}{[ K / P ]} {\sum }_{i = 1}^{[ K / P ]} {Z}_{i} ( 1 ) \\ and & ( 11 ) \\ {σ}_{z}^{2} = \frac{1}{[ K / P ]} {\sum }_{i = 1}^{[ K / P ]} {( {Z}_{i} ( 1 ) - {m}_{z} )}^{2} & ( 12 )\end{matrix}$$

Segments of length P that are either artifacts or significantly contaminated by artifacts have a significant different value of the metric Z(1) compared to the average and are, therefore, considered “non-normal”. Based on experience with real heart beat pulse data, metrics Zᵢ(1) that are greater or smaller than the average by at least one standard deviation are considered as “non-normal”, i.e. a segment zᵢ(j), with Zᵢ(1): |Zᵢ(1)−m_{z}|≧σ_{z} is designated as “non-normal”—box 312. Of course, it is possible to select other criteria for determining whether a segment is “normal” or “non-normal”. Finally, in the signal y(k) “non-normal” segments are replaced by the average m_{z}—box 314, while “normal” segments are left unchanged for producing a post-processed signal yₚₒₛₜ(k).

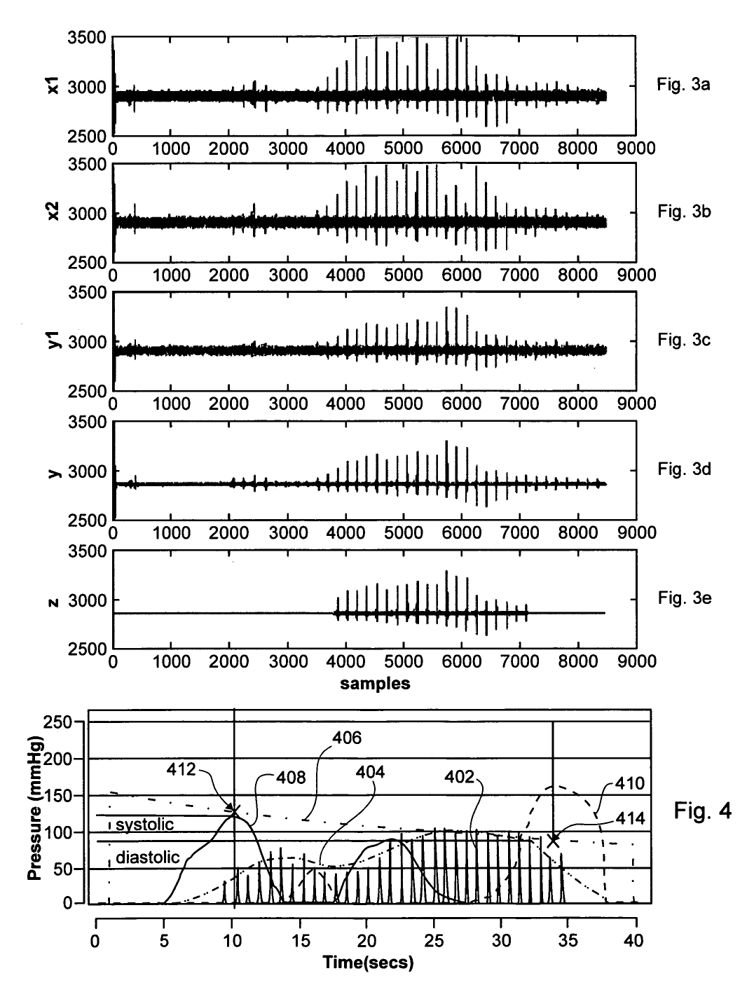

Referring to FIGS. 3a to 3e, signals after the different stages of the above signal processing for a two sensor implementation are shown. FIGS. 3a and 3b illustrate the digitized primary acoustic sensor signals x₁(k) and x₂(k). FIG. 3c illustrates the signal y₁(k) after beamforming and combining—block 202. FIG. 3d illustrates the signal y(k) after AIC processing—block 204, and FIG. 3e illustrates the signal after post-processing for artifact reduction—block 206. As is evident, the above signal processing according to the invention provides substantial improvement of the quality of the Korotkoff sound signal sensed by a two sensor implementation of the device 100.

Finally, pulse rate and blood pressure are determined in block 208 from the post-processed signal received from block 206 and a digitized pressure signal received from pressure transducer 114. The pulse rate is directly obtained from the post-processed signal at the output of block 206. Referring to FIG. 4, the Korotkoff signal 402, the envelope 404 of the Korotkoff signal, the cuff pressure 406, the positive derivative 408 of the envelope 404, and the negative derivative 410 of the envelope 404 are illustrated as a function of time. The systolic blood pressure is determined at the point 412 corresponding to the greatest magnitude of the positive derivative 408 of the signal's envelope 404. The diastolic blood pressure is determined at the point 414 corresponding to the greatest magnitude of the negative derivative 410 of the signal's envelope 404. The two points 412 and 414—indicating approximately the first and the last audible heartbeats—are referenced to the cuff pressure 406 of the deflating pressure cuff 102 as a function of time. The corresponding measured pressures are the systolic and the diastolic blood pressure.

The above method for signal processing is a preferred embodiment enabling highly accurate pulse rate and blood pressure measurements in environments comprising substantial noise and vibrations such as a helicopter or ambulance. As is evident, there are numerous other combinations of the above signal processing steps together with different embodiments of the device 100 implementable. In its simplest form, the device 100 comprises one primary acoustic sensor 110 and the signal processing comprises the processing steps of blocks 206 and 208, i.e. the digitized primary acoustic sensor signal x(k) is then directly provided to processing block 206 instead of signal y(k) for artifact reduction due to physiological motion of the patient. This embodiment provides a simple device and signal processing method for environments without substantial noise and vibrations such as a clinical setting or a doctor's office. An improved embodiment comprises an array of primary acoustic sensors 110, for example, an array of two sensors 110. Accordingly, the signal processing comprises blocks 202, 206, and 208, i.e. the digitized signals x₀(k),x₁(k), . . . x_{N}(k) of the plurality of sensors is beamformed and combined in block 202 and the output y₁(k) is then provided to block 206 for artifact reduction. The use of an array of primary acoustic sensors 110 together with the adaptive beamforming of block 202 substantially improves signal quality and accuracy of the measurements at the expense of a more complex device 100 and signal processing. The simplest form of the device 100 for use in environments with substantial noise and vibrations comprises one primary acoustic sensor 110 and the secondary acoustic sensor 112. Here, the corresponding signal processing comprises blocks 204, 206, and 208, i.e. the digitized primary acoustic sensor signal x(k) is directly provided to processing block 204 for AIC instead of the signal y₁(k). However, in very noisy environments such as a helicopter it is preferred to improve the signal quality by employing an array of primary acoustic sensors 110 and adaptive beamforming in order to obtain more reliable results.

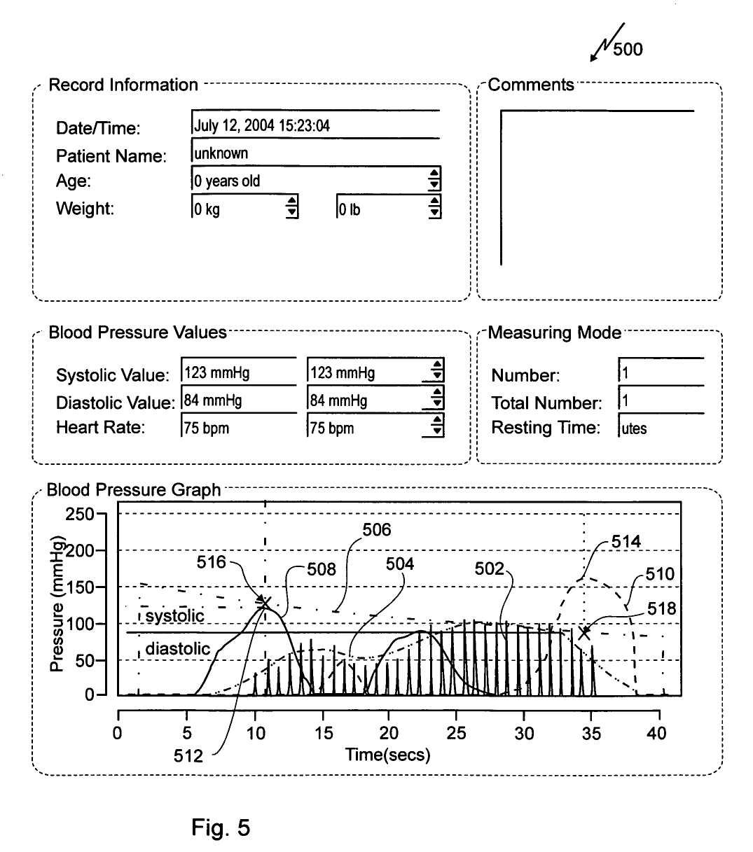

As described above, the automated blood pressure measurement system is based on the auscultatory method for determining heart pulse rate and systolic/diastolic blood pressure. It employs at least a primary acoustic sensor 110 for detecting Korotkoff sounds of a patient during blood pressure measurement in a same fashion as a medical practitioner uses a stethoscope for listening to a patient's Korotkoff sounds. The medical practitioner determines the systolic blood pressure at the instance when the first Korotkoff sound is heard during deflation of the pressure cuff by reading the pressure in the cuff using a mercury sphygmomanometer. Similarly, the diastolic blood pressure is determined at the instance when the last Korotkoff sound is heard by the medical practitioner. In order to enable a user of the automated blood pressure measurement system to “identify” the Korotkoff sounds in a same way as the user “hears” the Korotkoff sounds in the traditional auscultatory method, a graphical user interface according to the invention is provided in a further embodiment. Referring to FIG. 5, a preferred embodiment of a graphical user interface 500 according to the invention is shown. Here, a Korotkoff sound signal 502 sensed by at least a primary acoustic sensor 110 and processed using, for example, one of the above methods for processing the same is plotted versus a pressure deflation curve 506 as a function of time. In order to facilitate interpretation of the measurements a signal envelope 504 of the Korotkoff signal and its positive 508 and negative derivative 510 are also plotted in the same graph. Peak 512 of the positive derivative 508 indicates the time instance where the systolic blood pressure 516 is determined by the automated system. Similarly, peak 514 of the negative derivative 510 indicates the time instance where the diastolic blood pressure 518 is determined. As is evident, the time instances determined as the peak of the positive 508 and negative 510 derivative of the envelope 504 are in close proximity to the first and the last heart beat pulses in the Korotkoff signal, respectively. With the graphic user interface depicted in FIG. 5, a user trained in the traditional auscultatory method is enabled to graphically verify the systolic and diastolic blood pressure and heart pulse rate of each measurement. For example, a user is able to read the blood pressure values at any point in the graph by simply pointing the mouse to any data point displayed and clicking. The blood pressure value at this point is then displayed, for example, at the bottom of the graph as shown in FIG. 5. Optionally, the various curves discussed above are displayed in different colours further facilitating interpretation of the measurements.

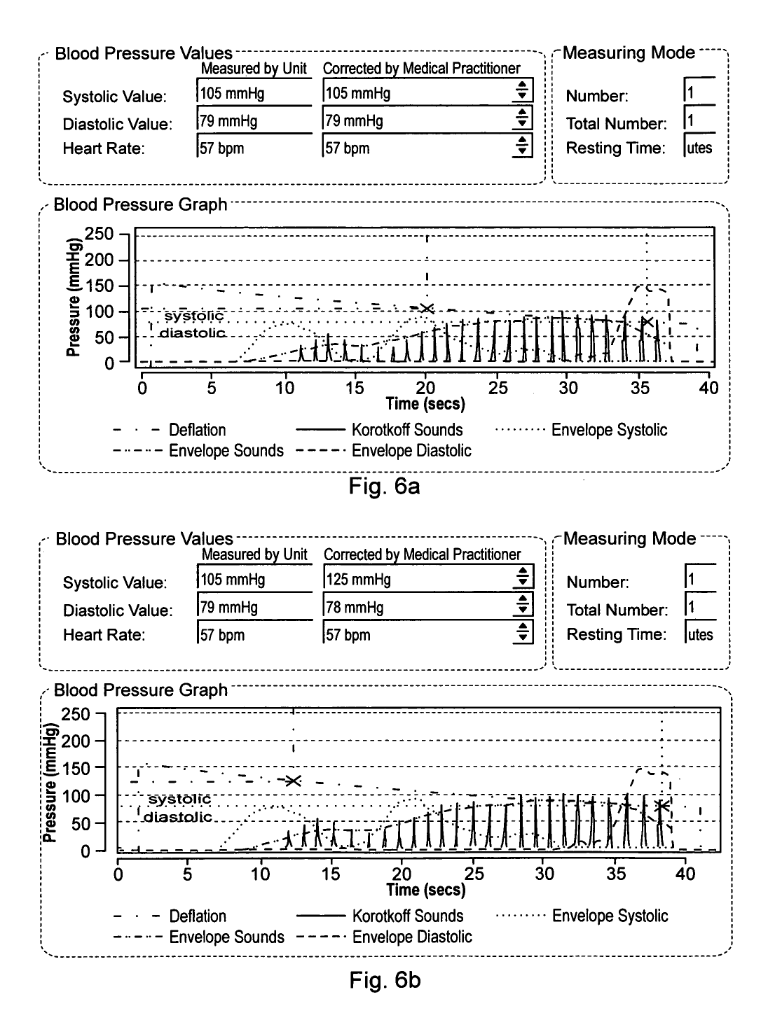

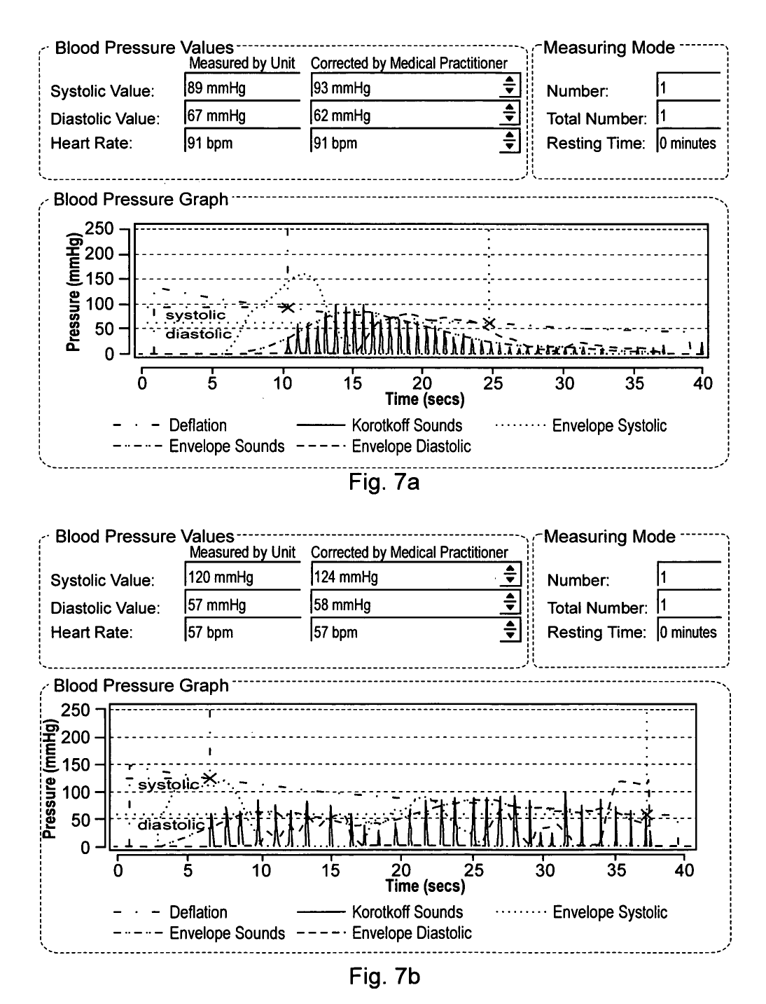

The graphical user interface 500 according to the invention is highly advantageous by enabling a user to correct an automatic measurement using his/her professional judgement as well as by helping the user to identify special patterns of heart beat such as Arrhythmia. Referring to FIGS. 6a and 6b, a case is illustrated where the automatic measurement missed the systolic blood pressure because of its derivative selection criteria, FIG. 6a. Upon viewing of the Korotkoff signal on the graphical user interface 500 the user will observe that the detected time instance is not in proximity to the first pulse of the Korotkoff signal and is able correct the systolic blood pressure by pointing the mouse to the time instance where the first pulse occurs as shown in FIG. 6b. Referring to FIGS. 7a and 7b, two cases of special heart beat patterns, Whistle—pulse train with long tail—indicative of a problem with heart valve contraction, and Arrhythmia, respectively, are illustrated. Graphically displaying the measured signals—Korotkoff sounds and deflation of cuff pressure—according to the invention enables a user trained in the traditional auscultatory method to detect various heart conditions such as Whistle and Arrythmia. Furthermore, by visually representing the traditional method the trained user is enabled to make a substantially more accurate diagnosis than by just “listening” to the Korotkoff sounds. By listening to the Korotkoff sounds it is much more difficult to detect variations in the heart beat period or the weak pulses of the Whistle while it is very easy to detect such conditions in the graphical display.

Furthermore, the graphical display of the Korotkoff sounds and deflation of cuff pressure enables a user to determine if a blood pressure measurement needs to be repeated. By looking at the graph the user is able to identify possible errors and decide whether a repeat measurement is needed, for example, in case of:

Underestimation of the systolic blood pressure because inflation pressure is too low;

Overestimation of the diastolic blood pressure because inflation pressure is too high;

Underestimation of the blood pressure because air tube is not connected properly;

Low heart rate signal because of improper placement of the cuff;

Pressure deflation is too fast/slow because of incorrect cuff size; and

Very noisy signal due to movement or improper placement of the cuff.

The combination of an automatic blood pressure measurement system based on the auscultatory method with the graphical user interface 500 according to the invention is highly advantageous by enabling a trained user to correct the automatic measurement, to decide whether a repeat measurement is necessary, and to quickly and accurately diagnose heart pulse patterns. These advantages are of particular importance in emergency situations where a trained user such as a paramedic has to act quickly and under extreme stress in order to save a patient's life.

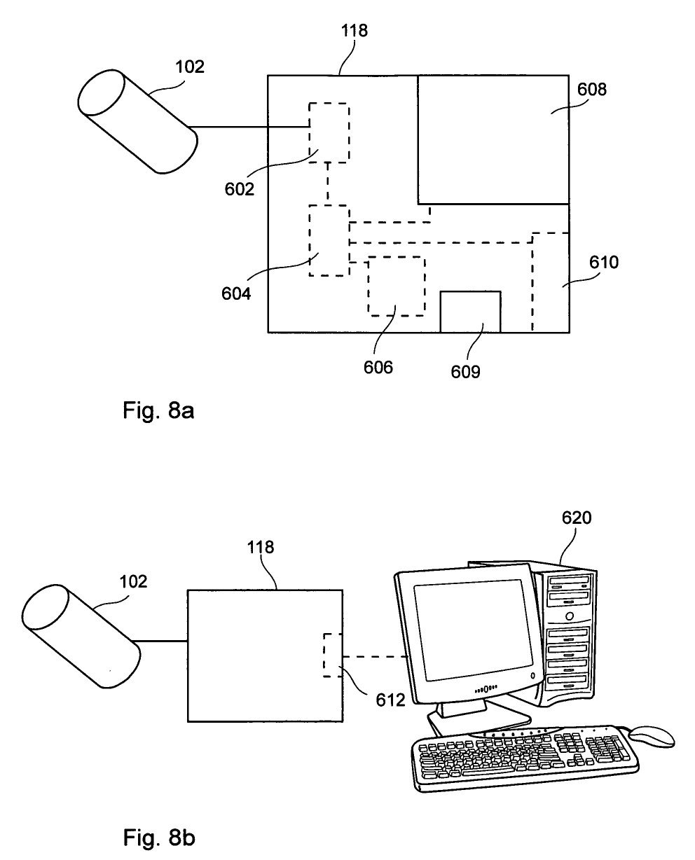

Referring to FIG. 8a, an embodiment of the automated blood pressure measurement device 100 according to the invention is shown. The housing 118 comprises, for example, an A/D converter 602 in communication with processor 604. Digitized signals are received by the processor 604 and processed using one of the signal processing methods outlined above. Execution of the processing steps is performed as a hardware implementation or by executing executable commands stored in memory 606. The results are then displayed using display 608 in communication with the processor 604. The display is implemented as a simple digital display for displaying numerical values or, alternatively, as a graphical user interface as outlined above. Preferably, the housing 118 also comprises a rechargeable battery 609 to increase mobility. Optionally, the housing 118 comprises a port 610 for coupling a portable memory device, not shown. This allows a user to store measurement results and/or transfer the same to a workstation for further processing such as statistical processing in long term monitoring of a patient or for transferring the results into a database. Alternatively, the housing 118 comprises a transmitter 612 for transmission of the measurement results to a workstation 620 via, for example, a wireless communication link 622, as shown in FIG. 8b.

Numerous other embodiments of the invention will be apparent to persons skilled in the art without departing from the spirit and scope of the invention as defined in the appended claims.

What is claimed is:

1. A method for determining systolic and diastolic blood pressure of a patient comprising:
receiving Korotkoff data indicative of a Korotkoff sound signal sensed during deflation of a pressure cuff wrapped around a limb of the patient;
receiving pressure signal data indicative of cuff pressure during the deflation of the pressure cuff;
processing the received Korotkoff data comprising:
determining a basic period corresponding to a heart pulse period;
dividing the Korotkoff data into non-overlapping segments of a length corresponding to approximately the basic period;
determining for each segment a metric;
calculating a statistical indicator of the determined metrics;
comparing for each segment the metric of the segment to the statistical indicator producing a comparison result for each segment;
producing post-processed Korotkoff data by comparing for each segment the comparison result to a predetermined threshold and if the comparison result is above the predetermined threshold replacing the signal data of the segment with the statistical indicator; and,
determining the systolic and the diastolic blood pressure based upon the post-processed Korotkoff data and the received pressure signal data.

2. A method for measuring blood pressure of a patient as defined in claim 1 comprising removing a mean from the Korotkoff sound signal.

3. A method for measuring blood pressure of a patient as defined in claim 1 wherein the statistical indicator comprises an average of the determined metrics.

4. A method for measuring blood pressure of a patient as defined in claim 1 comprising providing an alarm signal indicating irregular heart beat of the patient when the process of determining a basic period fails.

5. A method for measuring blood pressure of a patient as defined in claim 1 comprising determining a heart beat rate based upon the post-processed Korotkoff data.

6. A method for measuring blood pressure of a patient as defined in claim 5 wherein the basic period is determined by calculating an autocorrelation function of the Korotkoff data and by finding a second major peak in the autocorrelation function.

7. A method for measuring blood pressure of a patient as defined in claim 6 wherein the metric corresponds to an average power of a first harmonic of the Korotkoff data.

8. A method for measuring blood pressure of a patient as defined in claim 7 wherein the threshold is one standard deviation.

9. A method for measuring blood pressure of a patient as defined in claim 1 wherein the Korotkoff data are in dependence upon primary acoustic sensor signals received from a plurality of primary acoustic sensors disposed for sensing the Korottkoff sound signal and wherein the processing of the Korotkoff data comprises adaptive linear beamforming.

10. A method for measuring blood pressure of a patient as defined in claim 9 comprising combining the plurality of primary acoustic sensor signals producing a combined primary acoustic sensor signal.

11. A method for measuring blood pressure of a patient as defined in claim 10 comprising:
receiving secondary acoustic sensor signal data in dependence upon noise and vibrations; and,
removing interference due to noise and vibrations from the combined primary acoustic sensor signal using adaptive interference canceling and the secondary acoustic sensor signal data.

12. A method for measuring blood pressure of a patient as defined in claim 9 comprising:
receiving secondary acoustic sensor signal data in dependence upon noise and vibrations; and,
removing interference due to noise and vibrations from each of the plurality of primary acoustic sensor signals using adaptive interference canceling and the secondary acoustic sensor signal data.

13. A method for measuring blood pressure of a patient as defined in claim 1 wherein determining the systolic and the diastolic blood pressure comprises:
determining an envelope of the Korotkoff data;
determining a positive and a negative derivative of the envelope;
determining a maximum of each of the positive and the negative derivative;
determining the systolic blood pressure by relating the maximum of the positive derivative to a corresponding point in the pressure signal data; and,
determining the diastolic blood pressure by relating the maximum of the negative derivative to a corresponding point in the pressure signal data.

14. A method for measuring blood pressure of a patient as defined in claim 13 comprising graphically displaying the Korotkoff data, the envelope of the Korotkoff data, the positive and the negative derivative of the envelope, and the pressure signal data in a same graph indicating points in the graph related to the determination of the systolic and diastolic blood pressure.

15. A method for measuring blood pressure of a patient as defined in claim 14 comprising receiving input data for determining a different value of at least one of the systolic and the diastolic blood pressure.

16. A method for determining systolic and diastolic blood pressure of a patient comprising:
providing to a processor Korotkoff data indicative of a Korotkoff sound signal sensed during deflation of a pressure cuff wrapped around a limb of the patient;
providing to the processor pressure signal data indicative of cuff pressure during the deflation of the pressure cuff;
using the processor determining the systolic and the diastolic blood pressure based upon the Korotkoff data and the received pressure signal data; and,
using a display in communication with the processor plotting data based on the Korotkoff data and the pressure signal data in a same graph and indicating data points in the graph corresponding to the determined systolic and diastolic blood pressure.

17. A method for measuring blood pressure of a patient as defined in claim 16 wherein
determining the systolic and the diastolic blood pressure comprises:
determining an envelope of the Korotkoff data;
determining a positive and a negative derivative of the envelope;
determining a maximum of each of the positive and the negative derivative;
determining the systolic blood pressure by relating the maximum of the positive derivative to a corresponding point in the pressure signal data; and,
determining the diastolic blood pressure by relating the maximum of the negative derivative to a corresponding point in the pressure signal data.

18. A method for measuring blood pressure of a patient as defined in claim 17 comprising plotting the envelope of the Korotkoff data, the positive derivative of the envelope and the negative derivative of the envelope in the graph.

19. A method for measuring blood pressure of a patient as defined in claim 18 comprising receiving input data for determining a different value of at least one of the systolic and the diastolic blood pressure.

20. A storage medium having stored therein executable commands for execution on a processor, the processor when executing the commands performing:
receiving Korotkoff data indicative of a Korotkoff sound signal sensed during deflation of a pressure cuff wrapped around a limb of the patient;
receiving pressure signal data indicative of cuff pressure during the deflation of the pressure cuff;
processing the received Korotkoff data comprising:
determining a basic period corresponding to a heart pulse period;
dividing the Korotkoff data into non-overlapping segments of a length corresponding to the basic period;
determining for each segment a metric;
calculating a statistical indicator of the determined metrics;
comparing for each segment the metric of the segment to the statistical indicator producing a comparison result for each segment;
producing post-processed Korotkoff data by comparing for each segment the comparison result to a predetermined threshold and if the comparison result is above the predetermined threshold replacing the signal data of the segment with the statistical indicator; and,
determining the systolic and the diastolic blood pressure based upon the post-processed Korotkoff data and the received pressure signal data.

21. A storage medium as defined in claim 20 having stored therein executable commands for execution on a processor, the processor when executing the commands performing:
determining a heart beat rate based upon the post-processed Korotkoff data.

22. A storage medium as defined in claim 21 having stored therein executable commands for execution on a processor, the processor when executing the commands performing adaptive linear beamforming.

23. A storage medium as defined in claim 21 having stored therein executable commands for execution on a processor, the processor when executing the commands performing:
receiving secondary acoustic sensor signal data in dependence upon noise and vibrations; and,
removing interference due to noise and vibrations from the combined primary acoustic sensor signal using adaptive interference canceling and the secondary acoustic sensor signal data.

24. A device for measuring systolic and diastolic blood pressure of a patient comprising:
at least a primary acoustic sensor for being placed on skin of a limb of the patient over an artery occluded by applying pressure thereupon using a pressure cuff, each of the at least a primary acoustic sensor for producing a primary acoustic sensor signal in dependence upon Korotkoff sounds during deflation of the pressure cuff;
a pressure transducer for sensing the cuff pressure during the deflation of the pressure cuff and for providing a pressure signal in dependence thereupon;
a processor in communication with the at least a primary acoustic sensor and the pressure transducer, the processor for performing:
determining a basic period corresponding to a heart pulse period;
dividing the Korotkoff data into non-overlapping segments of a length corresponding to the basic period;
determining for each segment a metric;
calculating a statistical indicator of the determined metrics;
comparing for each segment the metric of the segment to the statistical indicator producing a comparison result for each segment;
producing post-processed Korotkoff data by comparing for each segment the comparison result to a predetermined threshold and if the comparison result is above the predetermined threshold replacing the signal data of the segment with the statistical indicator; and,
determining the systolic and the diastolic blood pressure based upon the post-processed Korotkoff data and the received pressure signal data.

25. A device for measuring systolic and diastolic blood pressure of a patient as defined in claim 24 comprising a secondary acoustic sensor in communication with the processor for sensing noise and vibrations in a surrounding environment of the patient and for providing a secondary acoustic sensor signal in dependence thereupon, the processor for:
removing interference due to noise and vibrations from the primary acoustic sensor signal using adaptive interference canceling and the secondary acoustic sensor signal data.

26. A device for measuring systolic and diastolic blood pressure of a patient as defined in claim 24 wherein the at least a primary acoustic sensor comprises an array of primary acoustic sensors wherein the processor performs adaptive linear beamforming.

27. A device for measuring systolic and diastolic blood pressure of a patient as defined in claim 24 comprising a transmitter in communication with the processor for providing a communication link to a workstation.

28. A device for measuring systolic and diastolic blood pressure of a patient as defined in claim 24 comprising a graphical display in communication with the processor for plotting a Korotkoff signal in dependence upon the primary acoustic sensor signal and the pressure signal in a same graph and indicating points in the graph related to the determination of the systolic and diastolic blood pressure.

29. A device for measuring systolic and diastolic blood pressure of a patient as defined in claim 28 wherein the graphical display comprises a graphical user interface.

30. A device for measuring systolic and diastolic blood pressure of a patient comprising:
a primary acoustic sensor for being placed on skin of a limb of the patient over an artery occluded by applying pressure thereupon using a pressure cuff, the primary acoustic sensor for producing a primary acoustic sensor signal in dependence upon Korotkoff sounds during deflation of the pressure cuff;
a pressure transducer for sensing the cuff pressure during the deflation of the pressure cuff and for providing a pressure signal in dependence thereupon;
a processor in communication with the primary acoustic sensor and the pressure transducer for receiving primary acoustic sensor data in dependence upon the primary acoustic sensor signal and pressure signal data in dependence upon the pressure signal, the processor for performing:
determining the systolic and the diastolic blood pressure based upon the primary acoustic sensor data and the pressure signal data;
determining graphical data for plotting the primary acoustic sensor data and the pressure signal data in a same graph and indicating points in the graph related to the determination of the systolic and diastolic blood pressure; and,
a display in communication with the processor for receiving the graphical data and displaying the same.

31. A device for measuring systolic and diastolic blood pressure of a patient as defined in claim 30 wherein the graphical display comprises a graphical user interface.
